(19) United States
(12) Patent Application Publication
(10) Pub. No.: US 2022/0144702 A1
(43) Pub. Date: May 12, 2022

(54) LIVING STRUCTURAL MATERIAL

(71) Applicant: The Regents of the University of Colorado, a body corporate, Denver, CO (US)

(72) Inventors: Will V. Srubar, III, Boulder, CO (US); Sherrie Cook, Boulder, CO (US); Mija Hubler, Boulder, CO (US); Jeffrey Cameron, Erie, CO (US)

(73) Assignee: The Regents of the University of Colorado, a body corporate, Denver, CO (US)

(21) Appl. No.: 17/436,581

(22) PCT Filed: Mar. 4, 2020
(86) PCT No.: PCT/US2020/020863

§ 371 Date: Sep. 3, 2021

Related U.S. Application Data
(60) Provisional application No. 62/813,733, filed on Mar. 4, 2019.

(51) Int. Cl. C04B 24/00; C12N 1/20; C04B 14/06

(52) U.S. Cl. CPC C04B 24/00; C04B 2103/0001; C04B 14/06; C12N 1/20

(57) ABSTRACT

Disclosed are biopolymeric and biologically active mortars suitable for use in providing building materials having enhanced physical properties. Further disclosed are methods for making and using the disclosed materials.

### FEDERALLY SPONSORED RESEARCH

This invention was made with government support under grant number HR0011-17-2-0039 awarded by DOD/DARPA. The government has certain rights in the invention.

### FIELD

Disclosed are biopolymeric and biologically active mortars suitable for use in providing building materials having enhanced physical properties. Further disclosed are methods for making and using the disclosed materials.

### BRIEF DESCRIPTION OF THE FIGURES

FIG. 1 depicts the doubling time for one example of Synechococcus 7002 as determined from the number of colony forming units (CFU's) per gram of sand as measured during the growth phase.

FIGS. 2A-2C depict the formation of calcite from Synechococcus 7002. FIG. 2A is a control sample wherein no calcite is present. FIG. 2B depicts an area of abundant calcite formation derived from the mineralization of a sand-gelatin scaffold. The area in FIG. 2B in the red box is enlarged as FIG. 2C.

FIG. 3 demonstrates the viability of Synechococcus 7002 at 7, 14, and 21 days as a function of relative humidity as determined by the number of CFU's present per gram of sand in one embodiment. The measurements are mad at 50%, 75% and 100% humidity.

FIGS. 4A-4C are various views of a sample of the hybrid material disclosed herein. FIG. 4A is a side view of a representative sample. FIG. 4B is a top down view of the sample depicted in FIG. 4A using Chi a fluorescence. FIG. 4C represents a Chi a fluorescence bottom view of the same sample. The hybrid material was produced at 33% relative humidity. The Chi a images in FIGS. 4B and 4C indicate the presence of bacterial cell which possess the ability to undergo photosynthesis.

FIG. 5 is a plot of time versus the log of the number of CFU's per mL for styrene production using Escherichia coli-PAL2-FDC1 cultured in sand with 10% gelatin. As seen in FIG. 5, the doubling time for cell growth is approximately 0.55 hours.

FIG. 6 depicts the effect of relative humidity and time on the number of CFU's per gram of sand for the composition comprising Escherichia coli-PAL2-FDC1 cultured in sand with 10% gelatin. FIG. 6 shows the log CFU's/g sand at 33%, 50%, 75% and 100% humidity at 7, 14, 21 and 30 days.

FIG. 7 depicts the growth over 24 hours of E. coli-PAL2-FDC1 when treated with varying levels of an iragicure photoinitiator. Control is represented by (▪), 0.05 g/L is represented by (♦), 0.1 g/L is represented by (▴), 0.2 g/L is represented by (▾), and 0.3 g/L is represented by the purple (♦).

FIG. 8A-8B are photographs of disclosed hybrid composition bricks. FIG. 8A is the top view of a composite brick formed from E. coli-PAL2-FDC1 and FIG. 8B is a photograph of a 600 cm³ brick obtained by the disclosed process using Synechococus 7002 (15 cm×8 cm×5 cm).

FIGS. 9A-9C depict the formation of calcite from Escherichia coli HB101/pBU11. FIG. 9A is a control sample wherein no calcite is present. FIG. 9B depicts an area of abundant calcite formation derived from the mineralization of a sand-gelatin scaffold. The area in FIG. 9B in the red box is enlarged as FIG. 9C.

FIGS. 10A-10C show the compressive strength increased with lower relative humidity and the strength is increased with the addition of Synechococcus 7002. Green bars represent a sample comprising Synechococcus 7002 and gray bars indicate controls. FIG. 10A shows the maximum stress in MPa at 7 and 30 days for 50%, 75% and 100% relative humidity and FIG. 10B shows the yield stress in MPa under the same conditions. FIG. 10C shows the yield stress of a sample comprising Synechococcus 7002 vs. control at 33% relative humidity after 7 days.

FIGS. 11A-11C show the compressive strength increased with lower relative humidity and the strength is increased with the addition of E. coli HB101/pBU11 versus urea media control. Blue bars represent a sample comprising E. coli HB101/pBU11 and gray bars indicate controls. FIG. 11A shows the maximum stress in MPa at 7 and 30 days for 50%, 75% and 100% relative humidity and FIG. 11B shows the yield stress in MPa under the same conditions. FIG. 11C shows the yield stress of a sample comprising E. coli HB101/pBU11 vs. control at 33% relative humidity after 7 days.

FIGS. 12A-12C show the compressive strength increased with lower relative humidity and the strength is increased with the addition of E. coli-PAL2-FDC1 versus MM1 control. Red bars represent a sample comprising E. coli-PAL2-FDC1 and gray bars indicate controls. FIG. 12A shows the maximum stress in MPa at 7 and 30 days for 50%, 75% and 100% relative humidity and FIG. 12B shows the yield stress in MPa under the same conditions. FIG. 12C shows the yield stress of a sample comprising E. coli-PAL2-FDC1 vs. control at 33% relative humidity after 7 days.

FIGS. 13A-13C depict the first steps in the formation of the disclosed materials. FIG. 13A represents the initial microbial inoculation in a medium at 37° C. FIG. 13B depicts the growth and precipitation of CaCO₃ after 12-24 hours. FIG. 13C depicts the gelation step wherein physical crosslinking occurs at approximately 20° C.

FIG. 14 is a photograph of the final material after dehydration of the gelation phase and a gelation sample prior to dehydration (bottom).

FIG. 15 is a drawing depicting the precipitation of calcium carbonate (calcite) by precipitating bacteria.

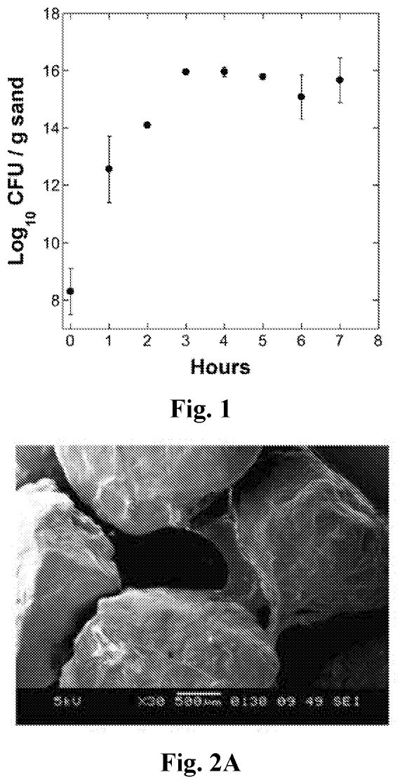

### DETAILED DESCRIPTION

The materials, compounds, compositions, articles, and methods described herein may be understood more readily by reference to the following detailed description of specific aspects of the disclosed subject matter and the Examples included therein.

Before the present materials, compounds, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

All percentages, ratios and proportions herein are by weight, unless otherwise specified. All temperatures are in degrees Celsius (° C.) unless otherwise specified.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

A weight percent of a component, unless specifically stated to the contrary, is based on the total weight of the formulation or composition in which the component is included.

“Admixture” or “blend” as generally used herein means a physical combination of two or more different components.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “hybrid” is used throughout the Specification to indicate that both inorganic and living components, i.e., microorganisms, comprise the building materials.

The term “nutrient” as used herein refers to any chemical compound or composition which provides for microorganism growth or function. For example, for calcium-precipitating bacteria, a source of calcium is a nutrient. For polymer-forming bacteria, glucose can be a nutrient which the bacteria converts to a polymeric material. Co-factors which support bacteria viability, for example, trace elements are considered nutrients.

### Mortars

Disclosed herein are living structural materials and mortars that are used to form and/or shape the living structural materials. The term “living” is applied to describe the mortars and building materials because during the fabrication of the building materials a living species, particularly microorganisms which provide a function in determining the final characteristics of the disclosed materials, are added. The mortars are uncured living structural materials. The mortars can be prepared at the location of use or they can be prepared at a facility wherein the mortars are shaped, for example, by filling in a mold. The mortars once shaped are then cured to form the disclosed building material.

In one aspect, the disclosed mortars comprise materials that are common to typical building materials, for example, bricks, concrete, and concrete patching materials. The term “mortar” as used herein is different from the use of the term in common building trades. For example, a brick wall is a structure wherein bricks are bound together by layers of mortar between the bricks. In the present disclosure the term “mortar” refers to the uncured material which when cured results in the living structural material disclosed herein.

In a further aspect, the disclosed mortars can comprise one or more recycled ingredients. This aspect relates to “shapeable” materials which can be manufacture in any configuration once cured. As such, the mortars do not necessarily comprise materials that are common to conventional building materials. Therefore, the disclosed structural materials can be used whenever a rigid structure is required. For example, as a replacement for plastic as used in the manufacture of an automobile, both interior and exterior.

The disclosed building materials have increased strength and decreased porosity. As such, the building materials are resistant to cracking or fracture. These enhanced properties are achieved by one or more of the aspects disclosed herein.

In one aspect, the disclosed living structural materials are formed by curing a mortar comprising:
a) one or more inert substrates;
b) one or more microorganisms; and
c) a nutrient medium.

As a result the resulting biopolymeric mortar resin wherein the bacteria (through precipitation mechanisms) produce a final material with both biological, i.e., living and structural, i.e., load bearing function.

In one aspect, the disclosed living structural materials are formed by curing a mortar comprising:
a) one or more inert substrates;
b) one or more microorganisms;
c) optionally one or more matrix forming elements; and
d) a nutrient medium.

In another aspect, the disclosed living structural materials are formed by curing a mortar comprising:
a) one or more inert substrates;
b) one or more precipitating microorganisms;
c) one or more matrix forming materials; and
d) a nutrient medium.

### Inert Substrates

The disclosed inert substrates can be any conventional material used to manufacture masonry. Non-limiting examples of inert substrates includes sand (SiO₂), porous and/or amorphous silica, for example, silica gel, gypsum (CaSO₄.2H₂O), calcium carbonate (CaCO₃), calcium oxide (CaO), alkali and alkali earth salts of silicate (SiO₄⁴⁻), for example, any orthosilicate, and clay. The disclosed clay can comprise quartz, metal oxides (Al₂O₃, MgO, and the like) as well as organic matter. The formulator can combine any of the disclosed inert materials, or others not listed, in any proportion desired to form an admixture suitable for use as an inert substrate.

### Precipitating Microorganisms

The disclosed precipitating microorganisms are microorganism, especially bacteria, which as a function of their biological processes are capable of converting organic or inorganic material to an insoluble substrate. Non-limiting examples of microorganisms that can induce the carbonate precipitation are photosynthetic microorganisms such as cyanobacteria and microalgae and sulfate-reducing bacteria. In one embodiment the precipitating microorganisms are chosen from Synechococcus sp. strain PCC 7002, Escherichia coli-PAL2-FDC1, Escherichia coli HB101/pBU11, Pseudomonas D2, Pseudomonas F2, Myxococcus xanthus, Bacillus sphaericus, Lysinibacillus sphaericus INQCS 414, and S. pasteurii MTCC 1761. One non-limiting example of a precipitating bacterium are species from the Synechococcus genus. Synechococcus is a unicellular cyanobacterium. Without wishing to be limited by theory, these bacteria perform biomineralization which produces insoluble material, for example, calcite.

In another embodiment, the disclosed materials are reinforced by precipitation of polymeric material within the interstices of the material prior to curing. For example, void volumes of the original admixture are filled with one or more biopolymers which are produced by one or more microorganisms. For example, a polystyrene-forming bacterium.

### Matrix Forming Materials

The disclosed matrix forming material is any organic material which is capable of enrobing the precipitating microorganisms in place such that the microorganisms can precipitate one or more materials which subsequently fills the interstices of the matrix forming materials and building material during curing. The matrix forming materials can also be combined with a source of carbon to form a matrix material. The matrix forming material can be pre-formed, for example, gelatin can be utilized or the material can be formed during curing.

A non-limiting example of a matrix forming material that is synthesized by a bacterium it the cellulose synthesized by Gluconacetobacter xylinus is in the form of organized, twisting ribbons, is a highly crystalline cellulose I allomorph. The glucan chains which are extruded from the cellulose synthesizing bacterium, are localized in the pores of the bacterial outer membrane and are associated outside into microfibrils and then into bundles of cellulose ribbons.

In another embodiment, Acetobacter xylinum and Acetobacter hasenii can be used to form cellulose fibrils. In one non-limiting example, precipitating bacteria and either Acetobacter xylinum or Acetobacter hasenii can be combined wherein the cellulose fibrils formed will entrain the precipitating bacteria which produce material which fills the interstices of the building material once cured. In this embodiment, glucose can be added via the herein below described nutrient medium as the source of carbon for the Acetobacter xylinum or Acetobacter hasenii. This embodiment can be summarized as follows:
a) one or more inert substrates;
b) one or more precipitating microorganisms chosen from Synechococcus sp. strain PCC 7002, Escherichia coli-PAL2-FDC1, Escherichia coli HB101/pBU11, Pseudomonas D2, Pseudomonas F2, Myxococcus xanthus, Bacillus sphaericus, Lysinibacillus sphaericus INQCS 414, or S. pasteurii MTCC 1761;
c) a matrix forming microorganism chosen from Acetobacter xylinum or Acetobacter hasenii; and
d) an aqueous nutrient medium comprising glucose.

### Nutrient Medium

The disclosed nutrient media comprise ingredients which provide for microorganism growth, as well as, the flowability of the mortar. Microorganism growth materials include inorganic salts and sources of carbon for microorganism metabolism.

Non-limiting examples of inorganic ingredients include NaCl, KCl, MgSO₄, CaCl₂, NaNO₃, KH₂PO₄, H₃BO₄, ZnCl₂, MoO₃, MnCl₂, CuSO₄, and CoCl₂. Non-limiting examples of organic ingredients include tris(hydroxymethyl)aminomethane and salts thereof, ethylenediaminetetraacetic acid and salts thereof, glucose, galactose, fructose and the like. Also included are matrix forming ingredients, for example, gelatin.

To insure flowability of the mortar, a carrier, for example, water is present which also serves to solubilize the ingredients that are water soluble.

Without wishing to be limited by theory the following embodiment is provided. A mortar comprising:
a) from about 65% to about 85% by weight of an inert substrate;
b) from about 1×10⁶ to about 1×10¹⁰ cells of a precipitating microorganism;
c) from about 1.5% to about 5% by weight of a matrix element; and
d) the balance a nutrient medium.

In one non-limiting iteration of this embodiment, the mortar comprises:
a) from about 65% to about 85% by weight of silicon dioxide, for example, washed and dried sand;
b) from about 1×10⁶ to about 1×10¹⁰ cells of a bacteria chosen from Synechococcus 7002, Escherichia coli-PAL2-FDC1, or Escherichia coh-HB101/pBU11;
c) from about 1.5% to about 5% by weight of a matrix element; and
d) the balance a nutrient medium.

In one non-limiting example of this iteration, the mortar comprises:
a) 75% by weight of sterilized and neutralized sand;
b) about 1×10⁸ cells of Synechococcus 7002;
c) 2.5% by weight of gelatin; and
d) the balance an aqueous nutrient medium comprising 2.52 mM CaCl₂).

In the above example, the nutrient medium can also comprise other nutrients, for example, MgSO₄, Na₂EDTA, KCl, and the like, as well as buffers.

Without wishing to be limited by theory, the mortars comprise bacteria which are capable of causing both organic and inorganic nutrients to agglomerate, condense or otherwise form a solid matrix within the interstices of the molded mortar as the mortar cures to form the disclosed building material. FIG. 15 is a drawing depicting the precipitation of calcium carbonate (calcite) by precipitating bacteria. In this example, calcium ions are taken up by the bacteria which subsequently combine the Ca²⁺ ions with carbonate (CO₃²⁻) to form the precipitated calcium.

In one embodiment the matrix element includes gelatin into which calcite can be enrobed once the mortar has cured. FIG. 15 illustrates this process. The depicted bacteria are enrobed within the matrix element, for example, gelatin.

### Biopolymeric Building Material

Once mortar has cured the bacteria no longer precipitate inorganic material. The formulator and user, however, can restore the microbiological activity of the building material by adding sufficient moisture and/or physical conditions to promote activity. In this way any surface imperfections formed during casting of the material, for example, into bricks, can be repaired after curing. This allows for reinforcement of the material to achieve the added strength that the cured material possesses.

Disclosed herein are materials which are stabilized or have increased resistance to fracture. The improvement lies in the action of one or more microorganisms that are present in the raw admixture of ingredients, i.e., the composition prior to introduction into a mold or shaped by methods known in the art.

As the mortar cures, the microorganism lose activity and ultimately become dormant. These microorganisms, however, can be reactivated by the application of a nutrient medium. In this way cracks or fissures which have formed can be filled by the existing microorganisms using the existing matrix elements and nutrient medium.

In one aspect, the disclosed biopolymeric building materials comprise:
a) one or more inert substrates;
b) one or more dormant microorganisms;
c) one or more matrix elements; and
d) one or more binding elements.

The disclosed binding elements are the combination of dormant microorganisms, nutrient materials, matrix elements and precipitated material.

As described herein above, the mortar as it cures forms binding elements, for example, an inorganic material. In one embodiment, the disclosed biopolymeric materials comprise a binding element that is calcite precipitated by a microorganism that formed within the interstices of the material as curing takes place. For example, void volumes of the original mortar are filled with precipitated calcium, i.e., calcite which is produced by one or more microorganisms.

For example, the disclosed materials comprise:
a) an inert inorganic substrate;
b) dormant microorganisms capable of precipitating an inorganic binder;
c) a matrix element; and
d) a binder.

In another embodiment, the disclosed materials are reinforced by precipitation of polymeric material within the interstices of the material prior to curing. For example, void volumes of the original admixture are filled with one or more biopolymers which are produced by one or more microorganisms. For example, a polystyrene-forming bacterium.

For example, the disclosed materials comprise:
a) sand;
b) one or more polymer precipitating microorganisms;
c) microorganisms that convert a source of carbon to a polymer; and
d) a binder.

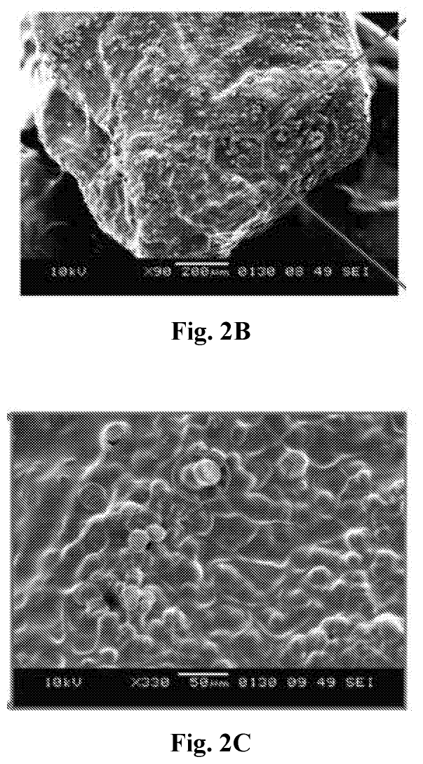

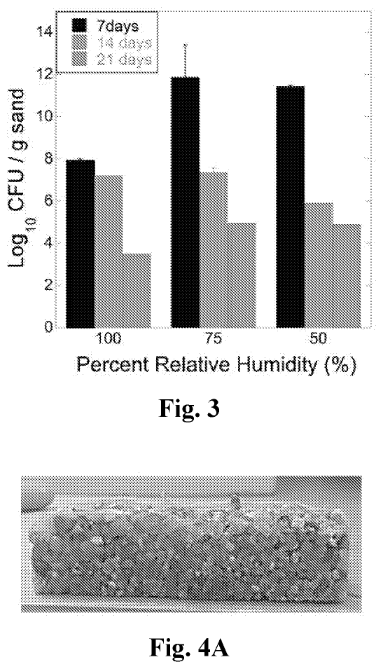

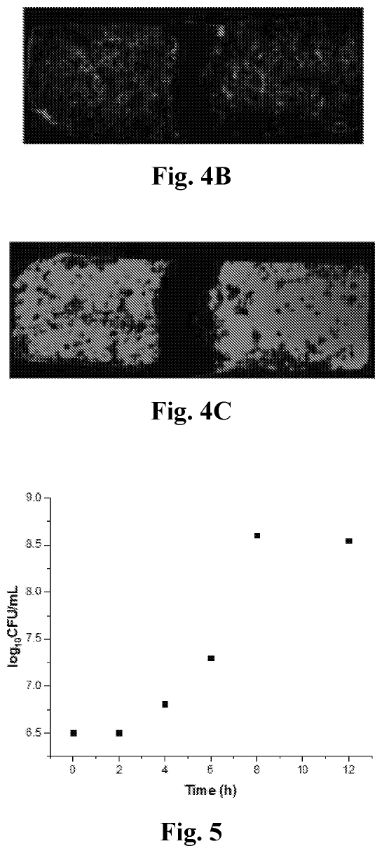

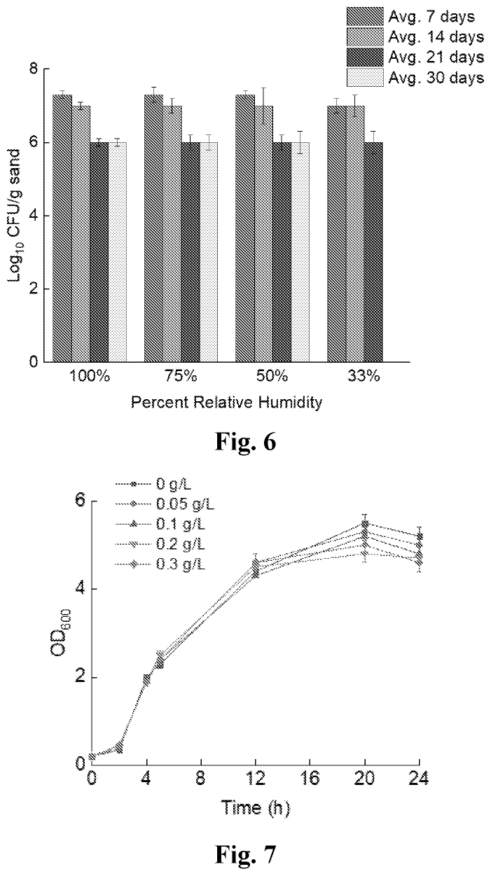

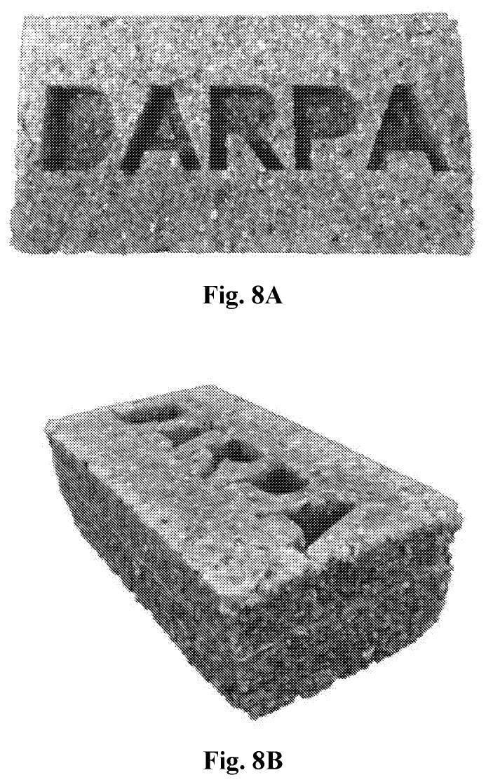

For example, FIGS. 2A-2C depict the formation of calcite from Synechococcus 7002. FIG. 2A is a control sample wherein no calcite is present. FIG. 2B depicts an area of abundant calcite formation derived from the mineralization of an example sand-gelatin scaffold. The area in FIG. 2B in the red box is enlarged as FIG. 2C. The result is the formation in situ of a binder (calcite) that enhances the thermal resistance, mechanical strength and buffer capacity of the material. In addition, because this microorganism is biologically active in the final product it provides a means for material self-repair.

One example of a crosslinking bacteria is Escherichia coli-PAL2-FDC1. This bacterium is capable of producing styrene from one or more disclosed nutrients, for example gelatin. The styrene can then be crosslinked by an optionally present polymerization photo initiator.

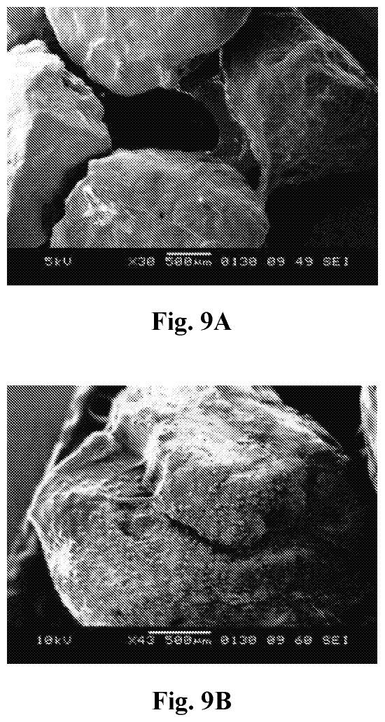

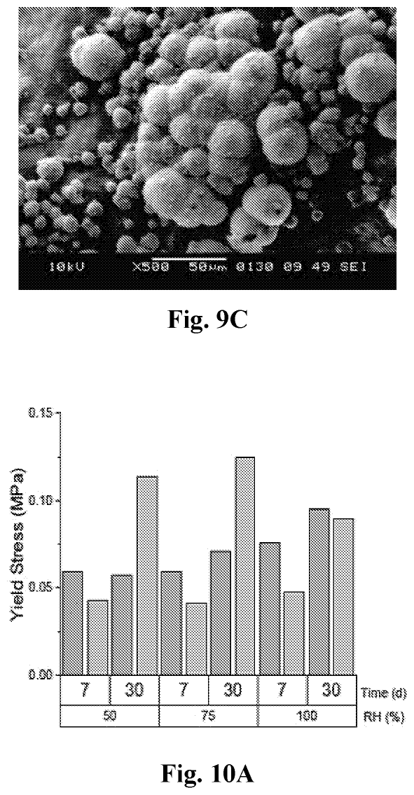

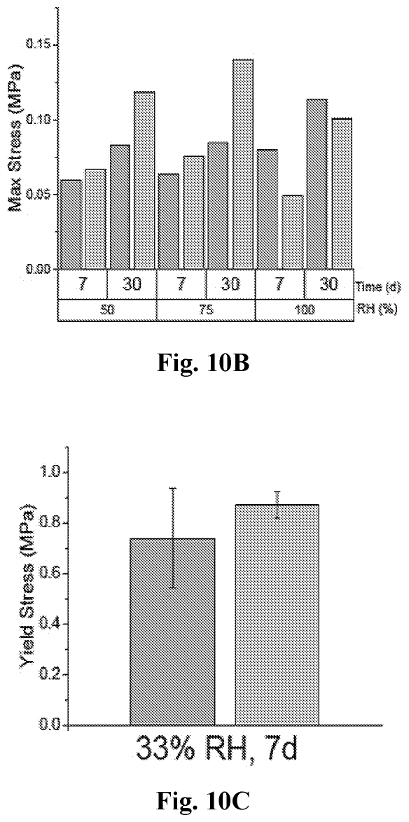

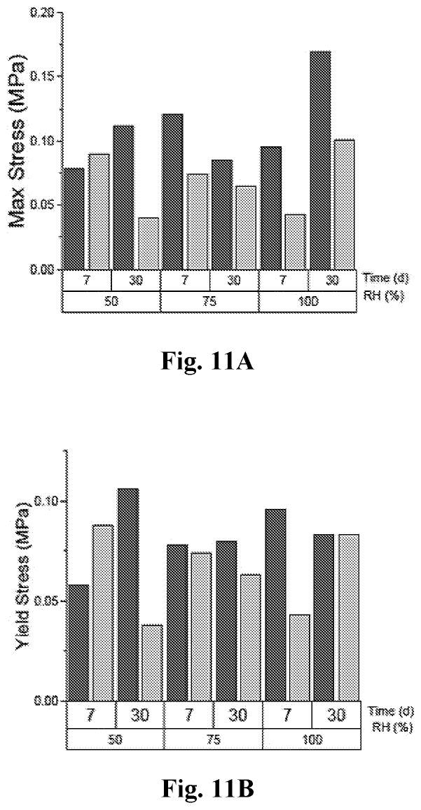

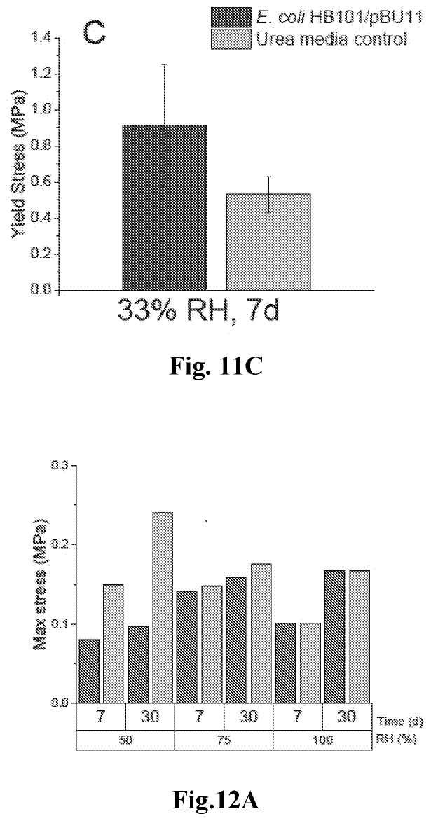

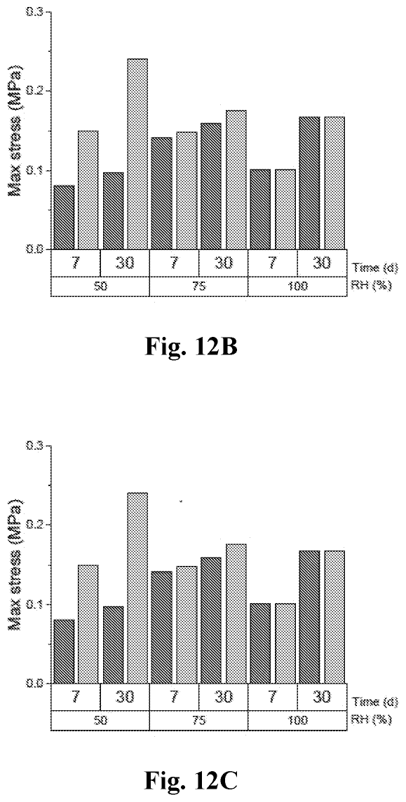

For example, FIGS. 9A-9C depict the formation of calcite from Escherichia coli-HB101/pBU11. FIG. 9A is a control sample wherein no calcite is present. FIG. 9B depicts an area of abundant calcite formation derived from the mineralization of a sand-gelatin scaffold. The area in FIG. 9B in the red box is enlarged as FIG. 9C. The result is the formation in situ of a binder (calcite) that enhances the thermal resistance, mechanical strength via the formation of polystyrene and buffer capacity of the material. In addition, because this microorganism is biologically active in the final product it provides a means for material self-repair.

The disclosed microorganisms act upon one or more nutrients to provide the increased compression and durability of the disclosed materials. In one aspect the reactive substrate is gelatin which the bacterial use to form crosslinked material which strengths to resulting material and decreases void volume. In another aspect the reactive substrate is mineralizing agent, for example, CaCO₃ which is precipitated by a bacterium to form calcite. In a further aspect, the reactive substrate is a cofactor which when acted upon by one or more bacteria, forms monomer which can be subsequently polymerized by the addition of a free radical initiator or by exposure to electromagnetic radiation.

### General Process

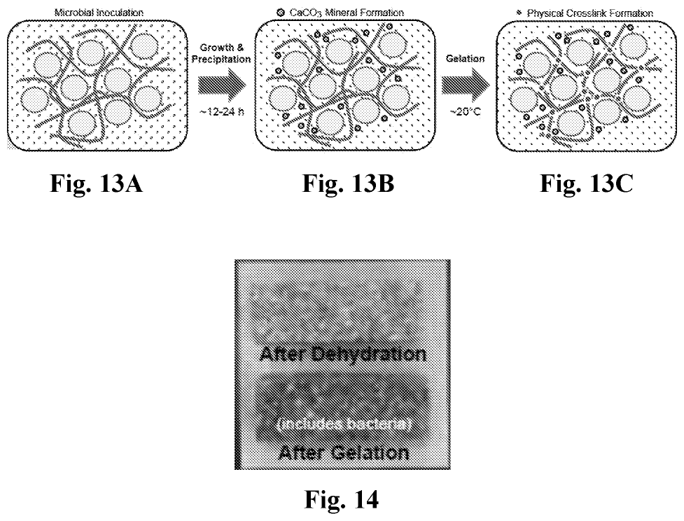

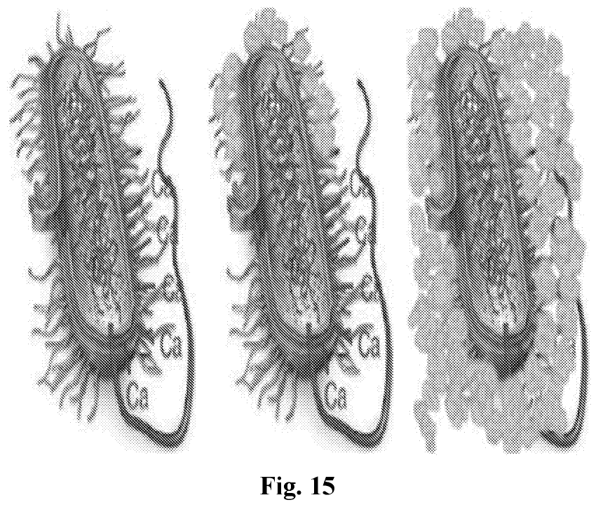

The following is a non-limiting example of the preparation and use of the disclose materials. Sand particles are saturated with a combination of water-based media that comprises microbial nutrients. Examples of nutrients include the compounds listed below in Tables I-IV. An amount of a biopolymer protein that is capable of being crosslinked, for example, gelatin is added. Once suitably combined, the admixture is inoculated with one or more bacterial culture, for example, a cyanobacteria. The admixture is incubated near physiological temperate (typically >30° C.). During this period, in one example as depicted in FIGS. 13A-13C and FIG. 14, the bacteria precipitate minerals that could add strength and reduce the bulk porosity of the mortar. In other aspects, gelatin can be replaced by polystyrene formed by styrene-precipitating bacteria, for example, Escherichia coli-PAL2-FDC1.

Once the microbial growth has plateaued the resulting admixture can be shaped by pouring into a mold. Reducing the temperature increases the level of gelation or other crosslinking thereby providing a structurally stable hybrid material. The formulator can adjust the process such that with a dehydration increase the material will achieve the strength of cement-based mortars, for example ˜500 psi.

The following is a non-limiting example of a nutrient medium. This medium is the commercially available A+ medium comprising the following ingredients. The final volume of this aqueous medium is 1000 mL.

To about 900 mL of distilled water is added in the following order:

TABLE I

| Component | Amount | | Conc. Stock Sol. | | Final Conc. | |
|---|---|---|---|---|---|---|
| NaCl | 18 | g | | | 0.308M | |
| MgSO₄ × 7H₂O | 5 | g | | | 0.02M | |
| Na₂EDTA × 2H₂O | 10 | mL | 3 | g/L | 0.08 | mM |
| KCl | 10 | mL | 60 | g/L | 8.05 | mM |
| CaCl₂ × 2H₂O | 10 | mL | 37 | g/L | 2.52 | mM |
| NaNO₃ | 10 | mL | 100 | g/L | 11.8 | mM |
| KH₂PO₄ | 10 | mL | 5 | g/L | 0.37 | mM |
| TRIS HCl pH 8.2 | 10 | mL | 100 | g/L | 8.26 | mM |
| Trace components* | 10 | mL | | | | |

The following is a listing of the Trace component concentrations

TABLE II

| Component | Conc. Stock Sol. | |
|---|---|---|
| H₃BO₃ | 55.5 | mM |
| ZnCl₂ | 230 | μM |
| MoO₃ | 21 | μM |
| ferric ammonium citrate | 300 | μM |
| MnCl₂ | 2.2 | mM |
| CuSO₄ | 1.2 | μM |
| CoCl₂ | 5 | μM |

The first eight components from Table I are combined in the specified order with continuous and efficient stirring. The total volume is than adjusted to 1000 mL by the addition of distilled water. The solution is then autoclaved.

A filtered solution of the Trace components from Table II is then added in the amount indicated. The solution is allowed to cool then refrigerated.

Another non-limiting example of an aqueous medium is NaCl free the A+ medium comprising the following ingredients. The final volume of this aqueous medium is 1000 mL.

To about 900 mL of distilled water is added in the following order:

TABLE III

| Component | Amount | | Conc. Stock Sol. | | Final Cone. | |
|---|---|---|---|---|---|---|
| MgSO₄ × 7H₂O | 5 | g | | | 0.02M | |
| Na₂EDTA × 2H₂O | 10 | mL | 3 | g/L | 0.08 | mM |
| KCl | 10 | mL | 60 | g/L | 8.05 | mM |
| CaCl₂ × 2H₂O | 10 | mL | 37 | g/L | 2.52 | mM |
| NaNO₃ | 10 | mL | 100 | g/L | 11.8 | mM |
| KH₂PO₄ | 10 | mL | 5 | g/L | 0.37 | mM |
| TRIS HCl pH 8.2 | 10 | mL | 100 | g/L | 8.26 | mM |
| Trace components* | 10 | mL | | | | |

The following is a listing of the Trace component concentrations

TABLE IV

| Component | Conc. Stock Sol. | |
|---|---|---|
| H₃BO₃ | 55.5 | mM |
| ZnCl₂ | 230 | μM |
| MoO₃ | 21 | μM |
| ferric ammonium citrate | 300 | μM |
| MnCl₂ | 2.2 | mM |
| CuSO₄ | 1.2 | μM |
| CoCl₂ | 5 | μM |

The first eight components from Table I are combined in the specified order with continuous and efficient stirring. The total volume is than adjusted to 1000 mL by the addition of distilled water. The solution is then autoclaved.

A filtered solution of the Trace components from Table IV is then added in the amount indicated. The solution is allowed to cool then refrigerated.

Optionally, one or more antibiotics or other additives can be added to the aqueous medium, for example, cyanocobalamin.

### Example 1

Purified sand (1 kg) is washed with 4% HCl for 24 hours followed by neutralizing with aqueous NaHCO₃ and water washings until the solution obtained is neutral. The sand is baked in an oven until dry and free flowing.

The media described in Tables III and IV (300 mL) is heated to 50° C. after which gelatin (30 g) is added with effective stirring until the solution is homogeneous. The mixture is then cooled to 40° C. after which 0.1 M NaHCO₃ is added, which is 2.52 g for 300 mL media. The solution is stirred at 40° C. and 0.1 M CaCl₂ is added, 3.33 g for 300 mL media. The pH is then checked. If the pH is above or below 7.6 then 3N HCl or 3N NaOH is added, respectively, to correct the pH. Cool the solution to 37-38° C. and add 1×10⁸ Synechococcus 7002 cells. In this example, 300 μL of a 4 μg/mL stock solution of vitamin B12 is added.

While incubating a light source is applied to the mold. Fluorescent lights are used to provide full spectrum wavelength with an intensity of 200 μmol m⁻² s⁻¹.

The gelatin-biocomposite materials require ‘curing’ before the final form is ready for usage. Curing is the process of dehydration to reach Equilibrium mass (i.e., all free water has evaporated). The material gains strength as it dehydrates. Curing can be accomplished in a range of environmental conditions. Molds can be cured in refrigerated (4° C.) or ambient conditions, at a range of relative humidity. At ambient temperatures less time is required to achieve a given structure. Similarly, lower relative humidity also imparts greater final strength to the biocomposite. Relative humidity is easy to control through placing biocomposite structures in sealed containers with hydrated salts (specific salts prescribe the chamber relative humidity).

If it is desired to re-use the biotic component for regenerating additional structures, this component can be reheated between 37-40° C. to melt the gelatin and free the biotic component from the sand. Temperatures above 40° C. should be avoided in order to not heat-kill the bacteria. Separating the biotic component from the structure can be accomplished by either adding warm liquid to the structure, or gently heating to entire structure. It is recommended to add new media to “refresh” the biotic component and promote viability. This process of regeneration allows for the propagation of the biocomposite from the initial biocomposite. The rate of regeneration differs between different bacteria.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A biologically active structural material, comprising:
a) one or more inert substrates;
b) one or more microorganisms; and
c) a nutrient medium.

2. The structural material according to claim 1, wherein the one or more inert substrates are selected from the group consisting of one or more of sand (SiO₂), silica gel, gypsum (CaSO₄.2H₂O) calcium carbonate (CaCO₃), calcium oxide (CaO), alkali and alkali earth salts of silicate (SiO₄⁴⁻), day, recycled materials, ground glass, plastic, wood fiber or recycled concrete.

3. (canceled)

4. (canceled)

5. The structural material according to claim 1, wherein the one or more microorganisms are selected from the group consisting of Synechococcus sp. strain PCC 7002, Escherichia coli-PAL2-FDC1, Escherichia coli HB101/pBU11, Pseudomonas D2, Pseudomonas F2, Myxococcus xanthus, Bacillus sphaericus, Lysinibacillus sphaericus INQCS 414, and S. pasteurii MTCC 1761, or mixtures thereof.

6. The structural material according to claim 1, wherein the microorganism is Synechococcus sp. strain PCC 7002.

7. (canceled)

8. The structural material according to claim 1, wherein the nutrient medium comprises an inorganic compound chosen from NaCl, KCl, MgSO₄, CaCl₂, NaNO₃, KH₂PO₄, H₃BO₄, ZnCl₂, MoO₃, MnCl₂, CuSO₄, or CoCl₂.

9. The structural material according to claim 1, wherein the nutrient medium comprises an organic compound chosen from tris(hydroxymethyl)aminomethane or salts thereof, ethylenediaminetetraacetic acid or salts thereof, glucose, galactose, fructose, or mixtures thereof.

10. The structural material according to claim 1, further comprising one or more matrix forming elements.

11. The structural material according to claim 10, wherein the matrix forming elements are precipitated by the one or more microorganisms.

12. The structural material according to claim 1, wherein the matrix forming elements comprise bacteria synthesized cellulose.

13. (canceled)

14. A biologically active mortar, comprising:
a) from about 65% to about 85% by weight of an inert substrate;
b) from about 1×10⁶ to about 1×10¹⁰ cells of a precipitating microorganism;
c) from about 1.5% to about 5% by weight of a matrix element; and
d) an aqueous nutrient medium.

15. The mortar according to claim 14, wherein the inert substrate is comprises one or more of sand (SiO₂), silica gel, gypsum (CaSO₄.2H₂O), calcium carbonate (CaCO₃), calcium oxide (CaO), alkali and alkali earth salts of silicate (SiO₄⁴⁻), clay, recycled materials, ground glass, plastic, wood fiber or recycled concrete.

16. (canceled)

17. (canceled)

18. The mortar according to claim 14, wherein the microorganism is chosen from Synechococcus sp. strain PCC 7002, Escherichia coli PAL2-FDC1, Escherichia coli HB101/pBU11, Pseudomonas D2, Pseudomonas F2, Myxococcus xanthus, Bacillus sphaericus, Lysinibacillus sphaericus INQCS 414, and S. pasteurii MTCC 1761, or mixtures thereof.

19. (canceled)

20. (canceled)

21. A biopolymeric building material, comprising:
a) one or more inert substrates;
b) one or more dormant microorganisms; and
c) one or more matrix elements.

22. The material according to claim 21, wherein the inert substrate selected from one or more of the group consisting of sand (SiO₂), silica gel, gypsum (CaSO₄.2H₂O), calcium carbonate (CaCO₃), calcium oxide (CaO), alkali and alkali earth salts of silicate (SiO₄⁴⁻), clay, recycled materials, ground glass, plastic, wood fiber or recycled concrete.

23. (canceled)

24. (canceled)

25. The material according to claim 21, wherein the dormant microorganism is chosen from Synechococcus sp. strain PCC 7002, Escherichia coli-PAL2-FDC1, Escherichia coli HB101/pBU11, Pseudomonas D2, Pseudomonas F2, Myxococcus xanthus, Bacillus sphaericus, Lysinibacillus sphaericus INQCS 414, and S. pasteurii MTCC 1761, or mixtures thereof.

26. The material according to claim 21, wherein the dormant microorganism is Synechococcus sp. strain PCC 7002.

27. The material according to claim 21, wherein the matrix element comprises an admixture of a polymeric material and a precipitated inorganic material.

28. The material according to claim 21, wherein the polymeric material is gelatin.

29. The material according to claim 21, wherein the precipitated inorganic material is calcite.

30. A method for forming a biopolymeric building material, comprising desiccating or drying a mortar according to claim 1.
